(19) United States
(12) Patent Application Publication
(10) Pub. No.: US 2005/0090825 A1
(43) Pub. Date: Apr. 28, 2005

(54) Bone plate

(75) Inventors: Joachim Pfefferle, Munstertal (DE); Oliver Blume, Munchen (DE); Hermann Zeuner, Freiburg (DE)

Correspondence Address: WELSH & KATZ, LTD, 120 S RIVERSIDE PLAZA, 22ND FLOOR, CHICAGO, IL, 60606, US

(73) Assignee: Medartis ag, Basel (CH)

(21) Appl. No.: 10/503,397

(22) PCT Filed: Feb. 15, 2002
(86) PCT No.: PCT/CH02/00094

(51) Int. Cl. A61B 17/56

(52) U.S. Cl. 606/69

(57) ABSTRACT

A bone plate is provided for treating a fracture, especially a fracture of an atrophic mandible, which bone plate comprises a plurality of eyelets through the holes of which screws can be screwed into the bone fragments in order to fasten the bone plate to the bone fragments so that the bone fragments on one side and on the other side of the fracture are maintained in a desired position relative to one another by means of the bone plate. The bone plate has a plate thickness in the range of from 0.5 mm to 1.6 mm, and comprises a stabilization zone intended to lie above the fracture, and the eyelets through the holes of which the screws can be screwed into the bone fragments are arranged on both sides of the stabilization zone, wherein the bone plate is designed as a single strip, and the stabilization zone is configured as a strip web whose width corresponds substantially to the external diameter of the eyelets and the moment of resistance of the web against bending of the bone plate in the plane of the plate is the same as or greater than the corresponding moment of resistance of the bone plate in the area between the eyelets.

### FIELD OF THE INVENTION

The invention relates to a bone plate for treating a fracture in accordance with the preamble of the independent patent claim.

### BACKGROUND

When treating fractures, especially fractures of an atrophic mandible which occur to an increasing extent in elderly patients, a great many problems may arise. Thus, for example, local factors such as inadequate blood supply caused by the frequent absence of the central artery, and sclerotic compacted bone stumps, present a risk in respect of consolidation. Moreover, the reduced cross section of the bone not only gives a small contact surface for the ends of the bone fragments on each side of the fracture, it also makes it difficult to achieve anatomically correct repositioning of the bone fragments. In cases of very severe atrophy, the bone near the fracture may be so thin that stable anchoring of larger osteosynthesis screws from larger systems (e.g. from what are called mandibular trauma and reconstruction plates) can only be achieved at a distance from the fracture. A compression of the fracture is thus impossible. In edentulous patients, application of splints is also not possible in practice. In conservative treatment methods, the permanent suprahyoid muscle tension on the edentulous fragments can be compensated only with difficulty. Furthermore, diseases which occur to an increasing extent with advancing age, for example osteoporosis, diabetes, kidney failure and many others, can also have an unfavorable effect on the course of healing. Moreover, the general state of health of elderly patients and the high morbidity risk entailed by prolonged immobilization limit the extent and the duration of surgical interventions.

Various therapeutic approaches have been employed to date. Of these, the conservative, noninvasive treatment methods now have a less important role. Two important approaches involve treating the fracture either with a miniplate or with a reconstruction plate.

Miniplates are bone plates which have a relatively small material thickness. These miniplates have the advantage that, in the surgical intervention, they can be introduced orally (i.e. through the mouth), and there is therefore no need to make a skin incision in the area of the patient's jaw. The miniplates can be easily bent to the shape of the bone fragments, but only in a few cases, if at all, is it possible to achieve a compression of the bone fragments in the area of the fracture. Because of their material thickness, however, miniplates have certain limitations in respect of the stability of the immobilization of the bone fragments in the area of the fracture, mainly in view of the fact that the patients are encouraged to start chewing again as soon as possible after the surgical intervention, thus causing considerable forces in the fracture area.

This is where the advantages of the reconstruction plates lie. These reconstruction plates are relatively solid bone plates (material thickness typically in the range of from 2.0 to 3.0 mm) which are very stable and can thus also take up large forces. Reconstruction plates, however, can be bent only with difficulty to fit the specific shape of the patient's mandible. Accordingly, reconstruction plates are also fastened with relatively solid, large screws which are screwed into the bone through corresponding eyelets provided on the bone plate, but this can only be done remote from the fracture if there is insufficient bone available. Thus, compression of the bone fragments in the fracture area is not possible, and, in addition to this, the reconstruction plates are often so large (precisely because they can be fastened only at a distance from the fracture) that they require a skin incision to be made in the jaw area in order to permit application of the reconstruction plate to the mandible.

### SUMMARY

Starting out from the aforementioned disadvantages of the existing bone plates, the object of the present invention is to propose a bone plate which on the one hand has a high degree of stability in the immediate vicinity of the fracture line and is also easy to bend (adapt) to the shape of the bone. Moreover, the dimensions of the bone plate are to be as small as possible in order to permit oral introduction of the bone plate in the surgical intervention and thus avoid a skin incision. Preferably, compression osteosynthesis ought also to be possible.

This object is achieved by a bone plate as is characterized by the features of the independent patent claim. Advantageous embodiments of the bone plate according to the invention are apparent from the features of the dependent patent claims.

In particular, the bone plate according to the invention has a plate thickness in the range of from 0.5 mm to 1.6 mm, and it comprises a stabilization zone which is intended to lie above the fracture. Arranged on both sides of this stabilization zone there are eyelets through the holes of which the screws can be screwed into the bone fragments. On the one hand, such a bone plate with such a stabilization zone permits stable immobilization in the immediate area of the fracture, and, on the other hand, the bone plate can be easily bent (adapted) to the shape of the bone fragments remote from the fracture. The plate thickness in this case particularly preferably lies in the range of from 0.8 mm to 1.2 mm. With such bone plates, it is possible to use screws of small dimensions to fasten the bone plate, and these screws can still be screwed in even if there is a small amount of bone available.

In one illustrative embodiment of the bone plate according to the invention, the stabilization zone is designed as a web. The bone plate can be designed in such a way that the moment of resistance of the stabilization zone or web against bending of the bone plate in the plane of the plate is the same as or greater than the corresponding moment of resistance of the bone plate in the area between the eyelets. The stabilization zone in this way acquires its stabilizing property.

The length of the web can be up to about six times the distance of the eyelets from one another. Such a web permits the good stability of the bone plate in the immediate area around the fracture line, but also allows the plate to be bent to shape in an area remote from the fracture. However, the dimensions (in particular the length) of the bone plate can be kept such that, in the surgical intervention, the plate can be introduced orally, so that no skin incision is required.

In an advantageous illustrative embodiment of the bone plate according to the invention, the outer contour of the web is oval. This outer contour has proven particularly advantageous in respect of the loads that have to be taken up, but it is of course also possible to consider other contours (e.g. contours which extend parallel to the longitudinal axis of the plate in the middle area of the web and then decrease linearly to a smaller width at the edge of the web, or contours which decrease linearly, directly from the middle of the web, to a smaller width at the edge of the web). Rectangular contours, in which the web thus has a uniform width practically up to its edge regions, can also be considered for example.

In an advantageous illustrative embodiment of the bone plate according to the invention, the width of the web corresponds substantially to the external diameter of the eyelets. Thus, the width of the bone plate too can be kept small, which makes it easier to introduce and fit the bone plate, without the stability of the bone plate suffering as a result.

In a further advantageous illustrative embodiment of the bone plate according to the invention, the width of the web decreases from the middle of the web to the edge regions of the web. The bone plate is therefore at its widest in the middle of the web, which is intended to lie directly above the fracture line. It is here that it should also be able to take up the greatest loads. Toward the outside, that is to say toward the edge regions of the web, the width of the web decreases, and here the loads that are to be taken up can also partially be taken up already by the bone fragments, without adversely affecting the healing process in the area of the fracture line.

In an advantageous embodiment of the bone plate according to the invention, it is possible, in the edge regions of the stabilization zone or web, to provide holes through which screws can be screwed into the bone fragments. The bone plate can then be fastened to the bone fragments relatively close to the fracture, with correspondingly smaller bone screws. The holes in the edge regions of the stabilization zone or web can be designed in particular as compression holes, or it is also possible to provide separate compression holes in addition to conventional through-holes in the edge regions of the stabilization zone or web.

Finally, in the edge regions of the stabilization zone or web, slits can be provided to facilitate bending of the bone plate to the shape of the bone fragments in these edge regions of the stabilization zone or web.

Furthermore, in the holes of the eyelets and/or in the holes in the edge regions of the stabilization zone or web, an engagement contour can be provided for blocking the bone plate at a stable angle by means of a blocking thread provided on the screw, said engagement contour allowing the screw to be screwed in at different angles. Holes with such engagement contours are known from WO-A-00/66012, for example. The provision of such an engagement contour is advantageous in several respects. On the one hand, there is no need for drill guides with which holes for the screws have to be drilled in advance. Instead, the holes can be drilled without a drill guide, or self-tapping screws can be screwed without a guide (i.e. without preliminary drilling) directly into the bone fragments, because the engagement contour permits introduction of the screws at different angles. At the same time, the engagement contour permits stable blocking, so as to rule out self-loosening of the screws. Although the connection of screw and plate can in principle be released again, a significantly greater torque has to be applied than in conventional threaded connections.

### BRIEF DESCRIPTION OF THE DRAWINGS

Further advantageous embodiments will become clear from the following description of illustrative embodiments of the bone plate according to the invention, the description being given with reference to the drawing, in which:

FIG. 1 shows a first illustrative embodiment of a bone plate according to the invention in a plan view;

FIG. 2 shows a longitudinal section, along line II-II in FIG. 1, through the illustrative embodiment of the bone plate according to FIG. 1;

FIG. 3 shows a second illustrative embodiment of a bone plate according to the invention in a plan view; and

FIG. 4 shows a third illustrative embodiment of a bone plate according to the invention in a plan view.

### DETAILED DESCRIPTION

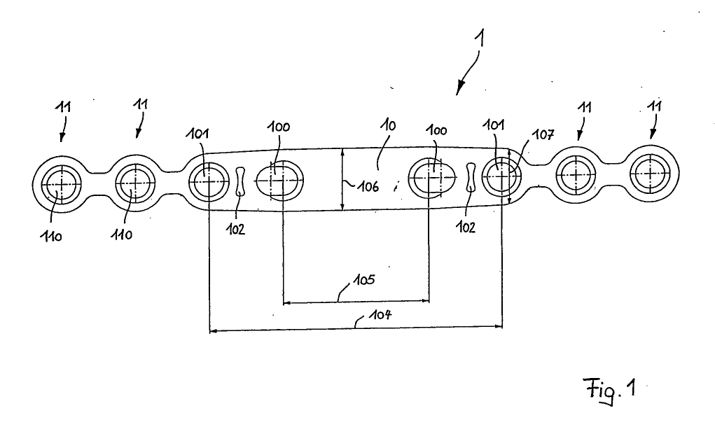

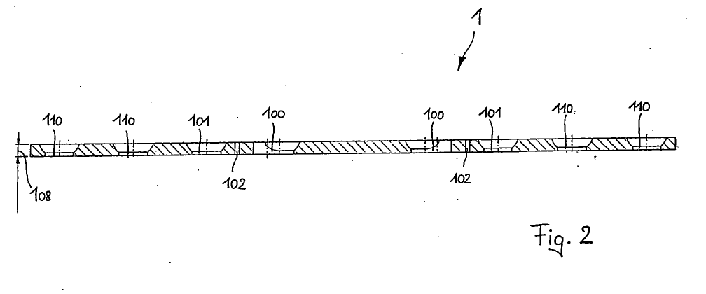

The first illustrative embodiment of the bone plate 1 according to the invention, shown in FIG. 1 and FIG. 2, comprises a stabilization zone, which is here designed as an oval web 10. Outside the web 10, on both sides thereof, eyelets 11 are provided (in the illustrative embodiment shown here, two eyelets 11 on each side) which for their part are connected via a narrow connecting web to one another and to the web 10 acting as stabilization zone and are provided for fastening the bone plate 1 in the area remote from the fracture. The holes 110 of the eyelets 11 can either be designed as threaded holes, or they can be designed such that, as is disclosed for example in WO-A-00/66012, they have an engagement contour for blocking at a stable angle. They can also have no thread at all, and the bone plate is fastened with the aid of bone screws which, apart from the bone thread, have no separate thread for engagement in a thread or engagement contour provided in the hole 110 of the eyelet. The bone plate is then fixed on the bone fragment in the area of the eyelet 11 by the screw head.

In each of its edge regions, the web 10 first has a compression hole 100. The way in which such compression holes 100 are designed and the way in which they function are known per se. They allow the ends of the bone fragments on each side of the fracture line to be compressed, which may prove necessary for the healing process (compression osteosynthesis).

The bone plate 1 shown in FIG. 1 also has, at each end, a through-hole 101 through which a conventional screw can be screwed. The through-hole 101 can be provided with a thread, or with an engagement contour as is known from WO-A-00/66012, into which a corresponding thread of the bone screw engages, but it can also be designed as a hole without a thread and bone screws are then used which, apart from the bone thread, do not have any separate thread for engagement in a thread or engagement contour provided in the through-hole 101. In this way, if appropriate after compression, the bone plate 1 can be fixed to the bone fragments relatively close to the fracture.

A slit 102 is provided in each case between the respective compression hole 100 and the directly adjacent through-hole 101. This slit 102 has the effect that, in the edge regions of the web 10, the bone plate already permits a certain bending of the bone plate to the shape of the bone fragments. However, the bone plate in the area of the slit 102 is still more stable against bending of the bone plate in the plane of the plate (greater moment of resistance) than in the area between two eyelets 110, and between the web 10 and the eyelet 110 directly adjacent to said web.

The length 104 of the web 10 (this is the distance 104 here, because the remaining part at the edge of the web 10 in each case in practice represents a half of an eyelet) is in this case about four times the distance between two adjacent eyelet holes 110, and thus four times the distance between two screws. The compression holes 100 are here at a distance 105 from one another which is twice the distance between two adjacent eyelet holes 110. Thus, between the two compression holes 100, a sufficiently large area of the web 10 remains for stable fixation of the ends of the bone fragments, and yet the dimensions of the bone plate 1 as a whole are kept to a limit.

The web 10 as a whole has an oval outer contour. Starting from the middle, where it has the width 106, its width decreases gently toward the edge regions, where it has the width 107, as a result of which, in the edge regions of the web 10, the bone plate can already be bent slightly easier to the shape of the bone fragments but still has a sufficient stability in respect of the immobilization in the area of the fracture line. Alternatively, other outer contours (as have already been mentioned above) may be considered. The width 106, 107 of the web 10 here corresponds substantially to the external diameter of the eyelets 11, which on the one hand ensures adequate stability of the bone plate 1 and on the other hand permits a uniform plate design, which is an advantage from the manufacturing point of view.

The thickness 108 of the plate (see FIG. 2) lies in principle in the range of from 0.5 mm to 1.6 mm, preferably in the range of from 0.8 mm to 1.2 mm, as is typical for miniplates, and it can very particularly be about 1.0 mm.

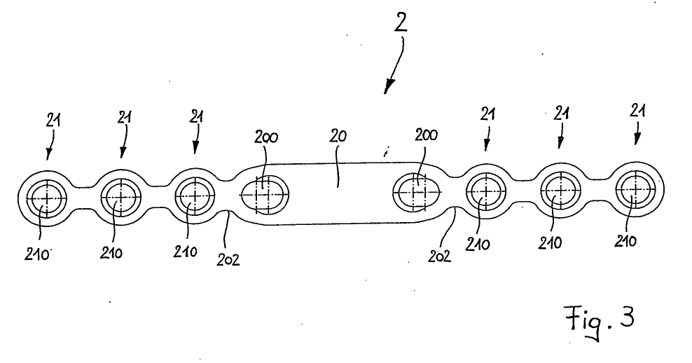

FIG. 3 shows a second illustrative embodiment of a bone plate 2 according to the invention in a plan view. The bone plate 2 shown in FIG. 3 has a web 20 which has a compression hole 200 at each of its end regions but, unlike the previously discussed bone plate, has no through-holes and also no slits. Instead, the bone plate 2 has three eyelets 21 at each end, and between the eyelet 210 situated closest to the respective compression hole 200 there is a web 202 which, although narrowed, is still wider than the webs between the other eyelets 210. This web 202 likewise permits slight bending of the bone plate in this area, but still has an increased stability against bending of the bone plate in the plane of the plate (so that this web 202 is to some extent comparable in terms of its function to the area with the slit 102 in the illustrative embodiment according to FIG. 1). As regards the eyelet holes 210, the same statements apply as have already been made above.

The web 20 is again intended to lie above the fracture line, the ends of the bone fragments can be immobilized in a stable manner by means of compression osteosynthesis in the area of the web 20. In the area of the eyelets 21 and of the narrow webs lying between the eyelets 21, the bone plate 2 can be bent easily to the shape of the bone fragments.

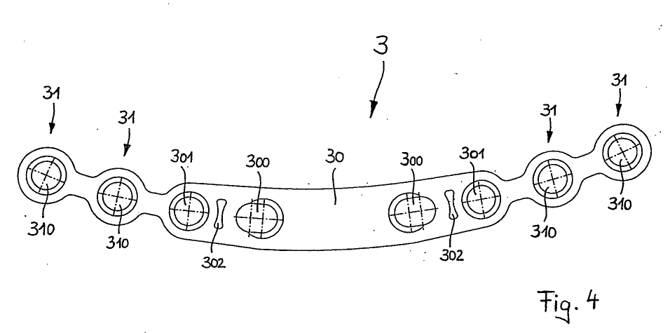

FIG. 4 shows a third illustrative embodiment of the bone plate 3 according to the invention in a plan view. This illustrative embodiment concerns an anatomically pre-shaped bone plate, the longitudinal axis of the plate describing a gentle arc. Because of its shape, the bone plate 3 is particularly suitable for use in the frontal region of the mandible.

Otherwise, the bone plate 3 essentially corresponds to the bone plate 1 described with reference to FIG. 1 and FIG. 2. Thus, as regards the web 30, the compression holes 300, the through-holes 301 in the web 30, the slits 302, and the eyelets 31 with eyelet holes 310, reference can be made to the corresponding parts of the description of the bone plate 1.

1-12. (canceled)

13. A bone plate for treating a fracture, especially a fracture of an atrophic mandible, which bone plate comprises a plurality of eyelets through the holes of which screws can be screwed into the bone fragments in order to fasten the bone plate to the bone fragments so that the bone fragments on one side and on the other side of the fracture are maintained in a desired position relative to one another by means of the bone plate, said bone plate having a plate thickness in the range of from 0.5 mm to 1.6 mm, and comprising a stabilization zone intended to lie above the fracture, and the eyelets through the holes of which the screws can be screwed into the bone fragments are arranged on both sides of said stabilization zone, wherein the bone plate is designed as a single strip, and the stabilization zone is configured as a strip web whose width corresponds substantially to the external diameter of the eyelets, and the moment of resistance of the web against bending of the bone plate in the plane of the plate is the same as or greater than the corresponding moment of resistance of the bone plate in the area between the eyelets.

14. The bone plate as claimed in claim 13, wherein plate thickness lies in the range of from 0.8 mm to 1.2 mm.

15. The bone plate as claimed in claim 13, wherein the length of the web is up to about six times the distance of the eyelets from one another.

16. The bone plate as claimed in claim 13, wherein the width of the web decreases from the middle of the web to the edge regions of the web.

17. The bone plate as claimed in claim 15, wherein the outer contour of the web is oval.

18. The bone plate as claimed in claim 13 wherein in the edge regions of the web, holes are provided through which screws can be screwed into the bone fragments.

19. The bone plate as claimed in claim 18, wherein the holes in the edge regions of the web are designed as compression holes.

20. The bone plate as claimed in claim 13, wherein in the edge regions of the web, slits are provided to facilitate bending of the bone plate in these edge regions of the web.

21. The bone plate as claimed in claim 13, wherein in the holes of the eyelets and/or in the holes in the edge regions of the web, an engagement contour is provided for blocking the bone plate at a stable angle by means of a blocking thread provided on the screw, said engagement contour allowing the screw to be screwed in at different angles.
